(19) United States
(12) Patent Application Publication
(10) Pub. No.: US 2025/0224389 A1
(43) Pub. Date: Jul. 10, 2025

(54) WATER QUALITY MEASUREMENT METHOD, DEVICE, EQUIPMENT, AND STORAGE MEDIUM

(71) Applicant: Guangdong University of Technology, Guangzhou (CN)

(72) Inventors: Qianqian CAI, Guangzhou (CN); Xiaopei CHEN, Guangzhou (CN); Chen ZHENG, Guangzhou (CN); Tong WANG, Guangzhou (CN); Damian MARELLI, Guangzhou (CN); Wei MENG, Guangzhou (CN)

(73) Assignee: Guangdong University of Technology, Guangzhou (CN)

(21) Appl. No.: 19/014,241

(22) Filed: Jan. 9, 2025

(30) Foreign Application Priority Data
Jan. 10, 2024 (CN) 202410036526.9

(51) Int. Cl. G01N 33/18; G06N 3/043

(52) U.S. Cl. CPC G01N 33/188; G01N 33/1813; G01N 33/1826; G06N 3/043; G06N 3/08

(57) ABSTRACT

This application discloses a water quality measurement method, device, equipment and storage medium, which includes: 1) Acquiring multiple sets of training data and using them to train the baseline network iteratively, calculating the error based on the predicted values output by the baseline network and the corresponding labels. 2) Calculating the error state value based on the errors obtained from two consecutive iterations. If the error state value of the current iteration meets the preset conditions, the parameters of the baseline network are updated with the error of the current iteration. Otherwise, the parameters are not updated. The process continues until the baseline network converges. 3) Using the model to obtain water quality measurement results of the wastewater treatment plant. This application addresses the issue present in the existing technologies where effective features from the raw dataset cannot be efficiently extracted, resulting in low accuracy of the measurement results.

### BACKGROUND OF THE INVENTION

With the increasing demand for better living environment, the discharge standards for wastewater are becoming increasingly strict. The concentration of effluent ammonia nitrogen (SNHe) and effluent total nitrogen (TN) are not only the most important parameters for water quality but also serve as the key indicators for the discharge standards of wastewater treatment processes (wastewater treatment plants). SNHe and TN are closely related with penalties caused by excessive emissions and are also intimately linked with the degree of water eutrophication. Therefore, it is of great necessity to monitor the concentration of SNHe and TN, as this can help to detect and effectively address the issue of water eutrophication in time. Currently, there are many methods to measure effluent ammonia nitrogen and total nitrogen, such as ultraviolet absorption spectrometry, ammonia nitrogen determination, step-by-step measurement of relevant elements followed by summation etc. Although these methods offer high precision, they are cumbersome, time-consuming, labor-intensive, and require specialized laboratory testing, which cannot meet the demands for real-time monitoring.

In contrast, data-driven soft measurement techniques can achieve rapid and accurate online predictions, overcoming the inherent limitations of chemical methods and online instruments measurements. Conventional data-driven methods normally exhibit weak data representation capabilities, as prediction models based on baseline networks typically rely on simple network structures, which cannot extract effective features from raw data collected by wastewater treatment plants. Besides, in many practical projects, raw data often contains noise and interference. In this case, the effective features of the dataset are crucial to ensuring that the baseline network model can accurately reflect the actual situation. Therefore, the lack of effective features often leads to a deterioration in the performance of the baseline network.

### SUMMARY OF THE INVENTION

The present application provides a water quality measurement method, device, equipment, and storage medium, aimed at solving the technical problem in the existing technologies where the effective features in the raw data sets cannot be efficiently extracted, leading to low accuracy in measurement results.

In this regard, the first aspect of the application provides a water quality measurement method, which includes:
Acquiring multiple sets of training data and determining the labels for each training data set. The training data includes nitrate nitrogen concentration, dissolved oxygen concentration, influent total nitrogen concentration, and influent suspended solids concentration, with the labels being either the effluent ammonia nitrogen concentration or the effluent total nitrogen concentration;
Training the baseline network iteratively using the training data, and calculating the errors based on the predicted values output by the baseline network and the corresponding labels;
Calculating the error state value based on the errors obtained from two consecutive iterations. If the error state value of the current iteration satisfies the preset conditions, then the network parameters of the baseline network will be updated using the error of the current iteration. If the error state value does not meet the preset conditions, the network parameters will not be updated. This process continues until the baseline network converges and the measurement model is obtained;
Conducting water quality measurements for the wastewater treatment plant by using the obtained measurement model.

Optionally, the method also includes:
Constructing the baseline network, which comprises an input layer, a membership function layer, a fuzzy rule layer, a normalization layer, and an output layer;

The iterative training of the baseline network includes:
Inputting the training data into the input layer of the baseline network;
Calculating the membership degree of the training data through the membership function layer;
Performing fuzzy processing on the membership degree of the training data through the fuzzy rule layer to obtain the fuzzy features of the training data;
Normalizing the fuzzy features of the training data through the normalization layer to obtain the normalized fuzzy features;
Performing the defuzzification of the normalized fuzzy features of the training data through the output layer to output the predicted values of the training data.

Optionally, the aforementioned calculation of the error state value based on the errors obtained from two consecutive iterations includes:
Calculating the deviation between the error obtained in the current iteration and the error obtained in the previous iteration to derive the first error state value for the current iteration;
Calculating the difference between the first error state value of the current iteration and the first error state value of the previous iteration to obtain the second error state value for the current iteration.

Optionally, the process of determining whether the error state value of the current iteration satisfies the preset conditions includes:
Determining whether both the first error state value and the second error state value of the current iteration are smaller than the preset threshold. If so, it can be concluded that the error state value of the current iteration satisfies the preset conditions. If not, it means that the error state value of the current iteration does not satisfy the preset conditions.

Optionally, the process of updating the network parameters of the baseline network using the error from the current iteration includes:
Calculating the learning rate for the current iteration based on the error of the current iteration;
Using the learning rate and the gradient of the current iteration to update the network parameters of the baseline network.

Optionally, the calculation of the learning rate for the current iteration based on the error obtained from the current iteration includes:
Calculating the L1-norm and L2-norm of the error of the current iteration;
Performing a weighted sum of the L1-norm and L2-norm of the error based on preset condition parameters to obtain the learning rate for the current iteration.

Optionally, the process of performing water quality measurement for the wastewater treatment plant using the measurement model includes:
Collecting the parameters from the wastewater treatment plant;
Inputting the parameters into the measurement model to predict the effluent ammonia nitrogen concentration or the effluent total nitrogen concentration, thereby obtaining the water quality measurement results of the wastewater treatment plant.

The second aspect of the application provides a water quality measurement device, which includes:
Data acquisition unit, which is used for acquiring multiple sets of training data and determining the corresponding labels for each set. Here, the training data includes nitrate nitrogen concentration, dissolved oxygen concentration, influent total nitrogen concentration, and influent suspended solids concentration, and the labels are effluent ammonia nitrogen concentration or effluent total nitrogen concentration;
Training unit, which is used for iteratively training a baseline network according to the training data, and calculating the error based on the predicted values output by the baseline network and the corresponding labels;
Parameter update unit, configured to calculate the error state value based on the errors from two consecutive iterations. If the error state value of the current iteration satisfies the preset conditions, then the network parameters of the baseline network will be updated using the error of the current iteration. If the error state value does not meet the preset conditions, the network parameters will not be updated. This process continues until the baseline network converges and the measurement model is obtained;
Measurement unit, which is used for performing water quality measurement for the wastewater treatment plant based on the measurement model and obtaining the water quality measurement results.

The third aspect of the application provides an electronic equipment, which includes a processor and a memory;
The memory is utilized to store and transmit the program code to the processor;
The processor is configured to execute any of the water quality measurement methods described in the first aspect according to the instructions in the program code.

The fourth aspect of the present application provides a computer-readable storage medium, which is used to store program code. When the program code is executed by a processor, it can implement any of the water quality measurement methods described in the first aspect.

From the above technical solutions, it can be seen that the present application has the following advantages:
The present application provides a water quality measurement method, which includes the following steps: acquiring multiple sets of training data and determining the labels for each set of training data. Here, the training data includes nitrate nitrogen concentration, dissolved oxygen concentration, influent total nitrogen concentration, and influent suspended solids concentration, with the labels being effluent ammonia nitrogen concentration or effluent total nitrogen concentration; iteratively training a baseline network using the training data, calculating errors based on the predicted values output by the baseline network and the corresponding labels; calculating an error state value based on the errors from two consecutive iterations, and updating the network parameters of the baseline network with the current iteration's error if the error state value satisfies the preset conditions. If the error state value does not satisfy the preset conditions, then the network parameters will not be updated. This process will continue until the baseline network converges. Using the measurement model to perform water quality measurement for the wastewater treatment plant and obtaining the water quality measurement results.

In this application, during the iterative training of the baseline network, the error state value is derived according to the errors in each iteration. And the error state value determines whether to update the network parameters or not. If the error state value does not satisfy the preset conditions, the network parameters will not be updated. In this way, it prevents the backpropagation of the abnormal data or the training data that may degrade the performance of the baseline network, ensuring effective feature extraction from the raw dataset and enhancing the validity of the data. This helps to improve the accuracy of the prediction results, and address the technical issues in the existing technologies where effective features from the raw dataset cannot be effectively extracted, leading to poor measurement accuracy.

### BRIEF DESCRIPTION OF DRAWINGS

To better illustrate the embodiments of the present application or the technical solutions in the existing technologies, the relevant figures will be briefly introduced below. It is evident that the figures described below are merely some embodiments of this application, and those skilled in the art can, without creative efforts, derive other figures based on these.

FIG. 1 is a flowchart illustrating a water quality measurement method provided in the embodiments of present application;

FIG. 2 is a structural schematic diagram of a water quality measurement system provided in the embodiments of the present application;

FIG. 3 is a schematic diagram of event definitions provided in the embodiments of the present application;

FIG. 4 is a comparative schematic diagram of RMSE (root mean square error) curves for SNHe modeling by using different networks provided in the embodiments of the present application;

FIG. 5 is a schematic diagram of network triggering situations for two event fusion mechanisms in SNHe modeling provided in the embodiments of the present application;

FIG. 6 is a comparative schematic diagram of the results of SNHe modeling by using different networks provided in the embodiments of the present application;

FIG. 7 is a schematic diagram of prediction errors for SNHe across different networks provided in the embodiments of the present application;

FIG. 8 is a comparative schematic diagram of RMSE curves for TN modeling by using different networks provided in the embodiments of the present application;

FIG. 9 is a schematic diagram of network triggering scenarios for two event fusion mechanisms in TN modeling provided in the embodiments of the present application;

FIG. 10 is a comparative schematic diagram of the results of TN modeling by using different networks provided in the embodiments of the present application;

FIG. 11 is a schematic diagram of prediction errors for TN across different networks provided in the embodiments of the present application;

FIG. 12 is a structural schematic diagram of a water quality measurement device provided in the embodiments of the present application.

### DETAILED DESCRIPTION OF THE INVENTION

To enable those skilled in the art to better understand the solution of this application, the technical solutions in the embodiments of this application will be clearly and comprehensively described below in conjunction with the attached figures. It is evident that the described embodiments are only a part of the embodiments of this application, rather than all of them. Based on the embodiments of this application, all other embodiments obtained by those ordinary skilled in the art without creative efforts shall fall within the protection scope of this application.

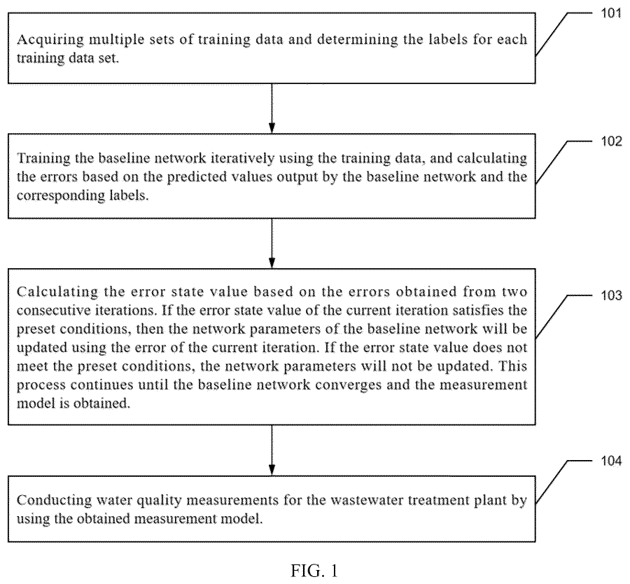

To facilitate understanding, please refer to FIG. 1. The embodiments of this application provide a water quality measurement method, comprising:

Step 101: Acquiring multiple sets of training data and determining the labels of each set of training data.

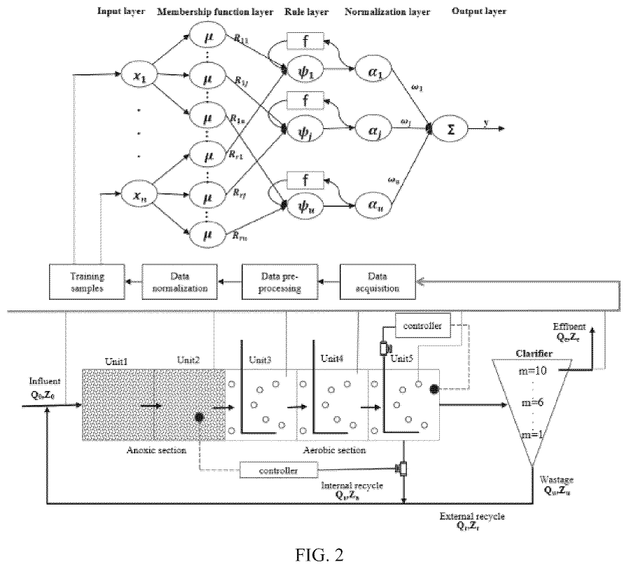

In the embodiments of this application, the training data includes nitrate nitrogen concentration, dissolved oxygen concentration, influent total nitrogen concentration, and influent suspended solids concentration, and the labels are effluent ammonia nitrogen concentration or effluent total nitrogen concentration. The data can be obtained from the Benchmark Simulation Model No. 1 (BSM1) which is jointly developed by the International Water Association and the European Union. The sampling interval can be 15 minutes, and the total collection period is 14 days. The collected data includes the nitrate nitrogen concentration (SNO2) in Unit 2 (FIG. 2), the dissolved oxygen concentrations (SO3, SO4, SO5) in Units 3, 4, and 5 (FIG. 2), influent total nitrogen concentration (TNin), influent suspended solids concentration (TSS), effluent ammonia nitrogen concentration, and effluent total nitrogen concentration.

When obtaining the soft measurement model for effluent ammonia nitrogen, the input data include SNO2, SO3, SO4, SO5, TNin, and TSS, and the output data (i.e., the label) is the effluent ammonia nitrogen concentration. As for the soft measurement model of effluent total nitrogen, the input data are SNO2, SO3, SO4, SO5, and TNin as well, with the output data (i.e., the label) being the effluent total nitrogen concentration. After determining the input and output data, the input data can be normalized.

Step 102: Training the baseline network iteratively using the training data, and calculating the errors based on the predicted values output by the baseline network and the corresponding labels.

A baseline network is constructed to receive the training data for iterative training. It should be noted that the baseline network can adopt the existing Convolutional Neural Network (CNN) structure. But in the embodiments of this application, a Recursive Fuzzy Neural Network (RFNN) is preferably used as the baseline network. As shown in FIG. 2, this baseline network consists of an input layer, a membership function layer, a rule layer, a normalization layer, and an output layer. During training, the training data Xᵢ=[x₁, x₂, . . . , xₙ]^{T} will pass through the input layer first and move to the next layer (i.e., membership function layer) directly. Here, xᵢ represents the i-th feature of the input data, which includes nitrate nitrogen concentration, dissolved oxygen concentration, influent total nitrogen concentration, and influent suspended solids concentration. The variable n denotes the dimensionality of the training data, i.e., the number of input features, here, n=5 in the embodiments.

The membership function layer is used to calculate the degree of membership of the training data, and each neuron in this layer represents a linguistic variable value. When the training data arrives at the membership function layer, the center c and width σ of the Gaussian membership function need to be updated during the iterative process. The membership function layer can be expressed as:

$${μ}_{i j} ( {x}_{i} ) = exp [ - \frac{( {x}_{i} - {c}_{i j} )}{{σ}^{2}} ] \qquad ( 1 )$$

where μᵢⱼ(xᵢ) represents the degree of membership of the i-th input feature xᵢ; cᵢⱼ is the center of the j-th Gaussian membership function for the i-th input feature.

In the fuzzy rule layer, each neuron represents a fuzzy rule. The fuzzy rule layer performs fuzzy processing on the membership degree of the training data, to obtain the fuzzy features of the training data. The fuzzy rule layer can be expressed as:

$${ψ}_{i} ( {x}_{i} ) = {f}_{i} {\prod }_{i = 1}^{n} {μ}_{i j} ( {x}_{i} ) \qquad ( 2 )$$

$${f}_{i} = \frac{1}{1 + exp ( - {h}_{i} )} \qquad ( 3 )$$

$${h}_{i} = {ψ}_{i} ( t - 1 ) {β}_{i} ( t ) \qquad ( 4 )$$

where ψᵢ(xᵢ) represents the fuzzy feature of the input feature xᵢ, f is the feedback function, h is an internal variable, ψᵢ(t−1) is the output at the (t−1)-th iteration of the fuzzy rule layer, and βᵢ(t) denotes the feedback weight of the recursive link at the (t−1)-th iteration;
The normalization layer normalizes the fuzzy features of the training data, to obtain the normalized fuzzy features of the training data. The normalization layer can be expressed as:

$${α}_{i} ( {x}_{i} ) = \frac{{ψ}_{i} ( {x}_{i} )}{{Σ}_{i = 1}^{u} {ψ}_{i} ( {x}_{i} )} \qquad ( 5 )$$

where αᵢ(xᵢ) represents the normalized fuzzy feature of the input feature xᵢ, and u denotes the number of fuzzy rules;
The output layer also serves as the defuzzification layer. It processes normalized fuzzy features of the training data through defuzzification, to output the predicted values. The output layer is:

$${y}_{i} = {\sum }_{i = 1}^{u} {ω}_{i} {α}_{i} ( {x}_{i} ) \qquad ( 6 )$$

where yᵢ represents the predicted value of the effluent ammonia nitrogen concentration or the effluent total nitrogen concentration corresponding to the training dataXᵢ, and ωᵢ denotes the output weight of the i-th fuzzy rule.

The error eᵢ is calculated based on the predicted value yᵢ output by the baseline network and the corresponding label ŷᵢ. In some example embodiments, the error can be obtained by calculating the difference between the predicted value and the label, i.e., eᵢ=yᵢ−ŷᵢ.

Step 103: Calculating the error state value based on the errors obtained from two consecutive iterations. If the error state value of the current iteration satisfies the preset conditions, then the network parameters of the baseline network will be updated using the error of the current iteration. If the error state value does not meet the preset conditions, the network parameters will not be updated. This process continues until the baseline network converges and the measurement model is obtained.

The deviation between the error obtained in the current iteration and the error obtained in the previous iteration is calculated to derive the first error state value corresponding to the current iteration. Specifically, a variable γ is defined to assess the trend of error reduction during the training process. By computing the mean error of the training data in the current iteration t, and calculating the difference between the mean error in the current iteration t and the mean error in the previous iteration t−1, the first error state value γ(t) for iteration t can be obtained as:

$$γ ( t ) = M E ( t ) - M E ( t - 1 ) \qquad ( 7 )$$

$$ME ( t ) = \frac{1}{N} {\sum }_{i = 1}^{N} ( {y}_{i} ( t ) - {\hat{y}}_{i} ( t ) ) \qquad ( 8 )$$

where yᵢ(t) represents the label of the i-th training data in the t-th iteration, ŷᵢ(t) represents the predicted value of the i-th training data in the t-th iteration, and yᵢ(t)−ŷᵢ(t) is the error of the i-th training data in the t-th iteration. ME(t) is the mean error of the t-th iteration, ME(t−1) is the mean error of the (t−1)-th iteration, and N is the number of training data input in the current iteration;
The second error state value κ(t) at the current iteration t is defined as the difference between the first error state value γ(t) at iteration t and the first error state value γ(t−1) at the previous iteration t−1, i.e., κ(t)=γ(t)−γ(t−1). The second error state value κ(t) represents the trend of error reduction.

Determining whether both the first error state value γ(t) and second error state value κ(t) of the current iteration are less than the preset threshold. If they are, it can be concluded that the error state value of the current iteration meets the preset condition; otherwise, it does not meet the preset condition.

Specifically, assuming that (γ(t), κ(t)) represents an event based on the error state, all events occurring during the training process of the baseline network can be defined as:

$$〈 \begin{matrix}\begin{matrix}\begin{matrix}{Event}_{1} = ( γ ( t ) > 0 , κ ( t ) > 0 ) \\ {Event}_{2} = ( γ ( t ) , κ ( t ) ) , oscillation\end{matrix} \\ {Event}_{3} = ( γ ( t ) < 0 , κ ( t ) > 0 ) )\end{matrix} \\ {Event}_{4} = ( γ ( t ) < 0 , κ ( t ) < 0 ) )\end{matrix} \qquad ( 9 )$$

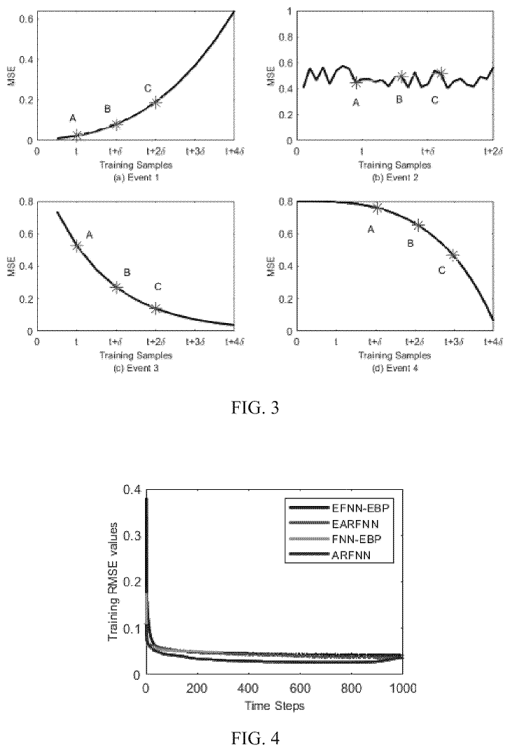

From FIG. 3, it can be observed that: 1) When (Event₁) occurs, it indicates that the error is increasing and exhibits an upward trend. 2) When (Event₂) occurs, it indicates that the error is fluctuating. 3) When (Event₃) occurs, it indicates that the error is decreasing, and the downward trend is becoming less apparent. 4) When (Event₄) occurs, it indicates that the error is decreasing, but the downward trend is becoming more evident.

Furthermore, it can be seen from FIG. 3 that when (Event₄) occurs, the training data becomes more effective, indicating that the network parameters need to be updated at this point. However, when other events occur, the training data at that moment may be ineffective, and the extracted features could be invalid. Therefore, no weight update is performed, meaning that for abnormal data or training data that degrades network performance, no backpropagation is conducted to update the network. This can effectively filter out abnormal data, thereby achieving the purpose of extracting effective features from the raw dataset and improving data validity. In other words, when (Event₁), (Event₂), or (Event₃) occurs, the network parameters are not updated, while when (Event₄) occurs, the network parameters are updated. The event-driven triggering mechanism proposed in the embodiments of this application can serve as a feature extractor and prevent the backpropagation of the data that may degrade the accuracy of the network (referred to as abnormal data), thereby enhancing the network's prediction accuracy.

After the training data passes through the aforementioned five layers, the forward propagation of the neural network is concluded. Subsequently, error backpropagation is required. Traditionally, the error backpropagation algorithm (EBP) based on gradient descent is employed, but this algorithm has a slow convergence rate and is prone to getting trapped in local optima, making it difficult to achieve the optimal solution. This will result in low prediction accuracy and long training durations. The Levenberg-Marquardt (LM) algorithm, as a typical second-order method, combines the advantages of the gradient descent method and Newton's method. This algorithm is characterized by fast convergence and high accuracy. The embodiments of this application improve the learning rate in the LM algorithm to further enhance the model's prediction accuracy.

In the embodiments, the update formulas for all network parameters Ω of the baseline network (such as center c, width σ, feedback weights β, and output weights ω) can be expressed as:

$$〈 \begin{matrix}{Event}_{4} : Ω ( t + 1 ) = Ω ( t ) - ( Q ( t ) + λ ( t ) ) \cdot g ( t ) \\ otherwise : Ω ( t + 1 ) = Ω ( t )\end{matrix} \qquad ( 10 )$$

where Q(t) is the pseudo-Hessian matrix at the t-th iteration, g(t) is the gradient vector at the t-th iteration, and λ(t) is the learning rate at the t-th iteration.

The value of λ(t) will affect the final water quality measurement result. To accelerate the learning process of the recursive fuzzy neural network, the embodiments of this application improve the learning rate by calculating the learning rate for the current iteration based on the error of the current iteration. Specifically, the L1-norm and L2-norm of the current iteration's error is computed. Then, a weighted sum of these norms is calculated using preset conditional parameters to obtain the learning rate for the current iteration:

$$λ ( t ) = θ \cdot {‖ e ( t ) ‖}_{2}^{2} + ( 1 - θ ) {‖ e ( t ) ‖}_{1} \qquad ( 11 )$$

In this equation, θ is the adjustment parameter, which can be set to 0.3; e(t)=[e₁(t), e₂(t), . . . , eₚ(t)] is the error vector at the t-th iteration, and p is the number of training data in the batch for the t-th iteration. In this learning rate, the introduction of the L1-norm and L2-norm as penalty factors enhances the model's adaptive learning capability. This allows to take larger learning steps when the error is significant, and take smaller learning steps when the error is minimal.

When the number of iterations of the baseline network reaches the maximum iteration count, or the error falls below the error threshold, or the error converges to a certain value, it is then determined that the baseline network has converged, and a trained measurement model is obtained.

After the training is completed, test data can be obtained from the Benchmark Simulation Model No. 1 (BSM1). This test data is then input into the trained measurement model to obtain the water quality measurement results. Subsequently, the measurement performance can be evaluated using the Root Mean Square Error (RMSE), Mean Absolute Percentage Error (MAPE), and Accuracy. The formulas for RMSE, MAPE, and Accuracy are as follows:

$$RMSE = \sqrt{\frac{1}{N} {\sum }_{i = 1}^{N} {( {y}_{i} - {\hat{y}}_{i} )}^{2}} \qquad ( 12 )$$

$$MAPE = \frac{1}{N} {\sum }_{i = 1}^{N} \frac{\vert {y}_{i} - {\hat{y}}_{i} \vert}{{y}_{i}} \qquad ( 13 )$$

$$Accuracy = \frac{1}{N} {\sum }_{i = 1}^{N} \frac{( 1 - \vert {e}_{i} \vert )}{\vert {y}_{i} \vert} \qquad ( 14 )$$

In the equation, eᵢ represents the error of the i-th test data.

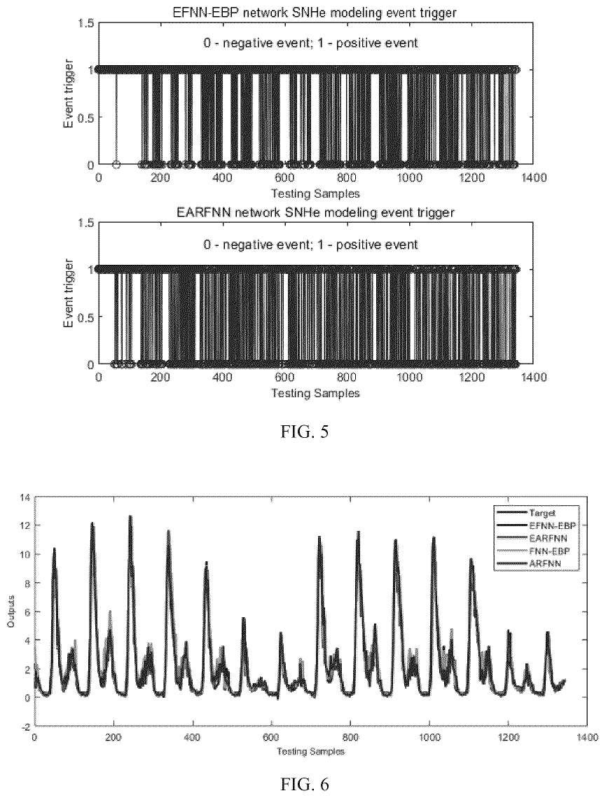

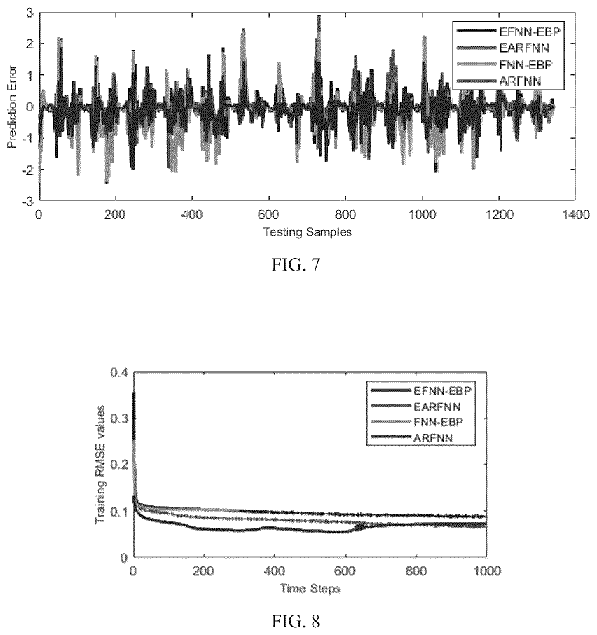

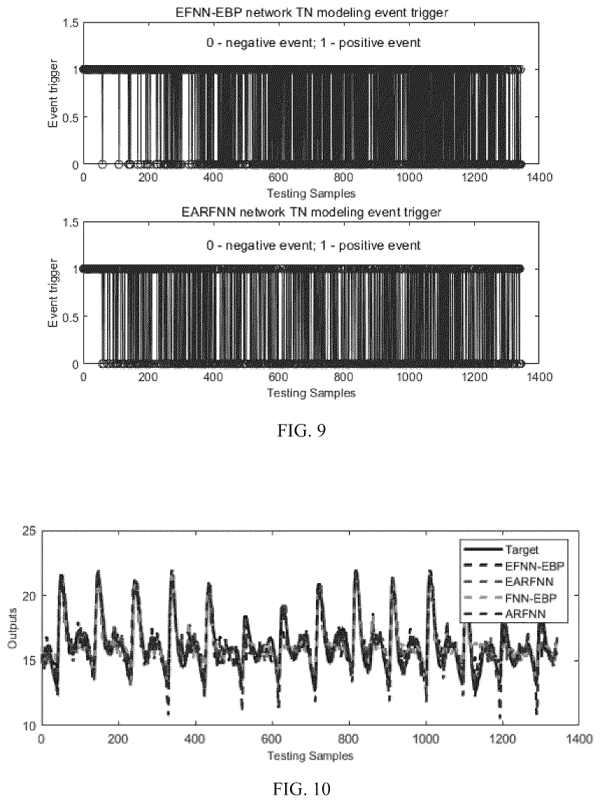

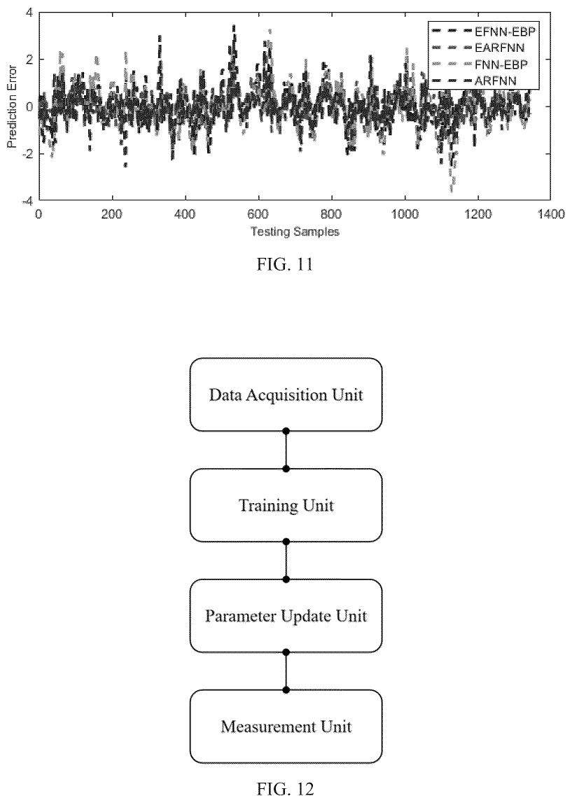

The embodiments of the present application verify the superiority of the proposed method through comparative experiments. First, soft measurements of effluent ammonia nitrogen concentration and effluent total nitrogen concentration are performed using a conventional EBP-based fuzzy neural network (FNN_EBP). Then, the event-driven triggering mechanism is incorporated into the FNN_EBP network, forming an event-driven EBP fuzzy neural network (EFNN_EBP) to validate the effectiveness of the event-driven triggering mechanism. Next, an improved adaptive recursive neural network (ARFNN) is employed to conduct the soft measurements of effluent ammonia nitrogen concentration and effluent total nitrogen concentration. Similarly, the event-driven triggering mechanism is added to the ARFNN network to form an event-driven ARFNN network (EARFNN). By comparing the soft measurement results of FNN_EBP, EFNN_EBP, ARFNN, and EARFNN networks, the superiority of the EARFNN model, which integrates the improved adaptive LM algorithm and event-driven mechanism, is demonstrated. Specific results can be found in FIGS. 4-11. The event-driven triggering mechanism proposed in the embodiments of this application serves as a feature extractor and prevent the backpropagation of the data that degrades the accuracy of the network (referred to as abnormal data), thereby enhancing the network's prediction accuracy. FIGS. 5 and 9 show the event triggering scenarios during the training of effluent ammonia nitrogen concentration and effluent total nitrogen concentration. By comparing FNN_EBP with EFNN_EBP and ARFNN with EARFNN, it is evident that the event-driven triggering mechanism can effectively enhance the accuracy of soft measurements and reduce errors.

The improved adaptive LM algorithm proposed in the embodiments of the present application exhibits faster convergence rate compared to the traditional EBP algorithm, as clearly reflected in FIGS. 4 and 8. Although the combination of ARFNN and the event-driven triggering mechanism sacrifices some convergence rate, the accuracy gets improved after each iteration, unlike ARFNN, where the accuracy tends to decrease as training progresses. This guarantees that the overall performance of the EARFNN model is superior to that of ARFNN.

Through the comparative experiments described above, it can be concluded that the ARFNN achieves the highest accuracy in soft measurements of effluent ammonia nitrogen concentration and effluent total nitrogen concentration, with the best overall performance in RMSE and MAPE. That is to say, the EARFNN model proposed in the embodiments, which integrates the event-driven triggering mechanism and adaptive LM algorithm, demonstrates significant competitiveness in soft measurement tasks.

The embodiments of the present application address the issue of low measurement accuracy caused by the inability to extract effective features from datasets in existing technologies. Specifically, it employs an event-driven triggering mechanism, which can effectively extract useful features from the raw data. For abnormal data or training data that degrades network performance, no backpropagation is performed to update the network, effectively filtering out the abnormal data. By doing so, it can achieve the purpose of extracting effective features from the raw dataset and enhancing data validity. Furthermore, to tackle the problems of slow convergence, susceptibility to local optima, and low measurement accuracy associated with the error backpropagation method used in existing neural networks, the embodiments of the present application adopt an improved Levenberg-Marquardt method instead of the error backpropagation method. By introducing the L1-norm and L2-norm as penalty factors in the learning rate, the model's adaptive learning capability is enhanced. This allows the model to take larger learning steps when the error is significant, and take smaller learning steps when the error is minimal, thereby improving the network's convergence rate, avoiding local optima, and enhancing the measurement accuracy.

Step 104: Conducting water quality measurements for the wastewater treatment plant by using the obtained measurement model.

Collecting water quality parameters from the wastewater treatment plant, which includes nitrate nitrogen concentration, dissolved oxygen concentration, inflow total nitrogen concentration, and inflow suspended solids concentration. Inputting water quality parameters into the measurement model to predict the effluent ammonia nitrogen concentration or effluent total nitrogen concentration, thereby obtaining the water quality measurement results of the wastewater treatment plant.

In the embodiments of this application, when iteratively training the baseline network using training data, the error state value is derived based on the errors from each iteration. And the error state value determines whether to update the network parameters or not. If the error state value does not satisfy the preset conditions, the network parameters will not be updated. In this way, it prevents the backpropagation of the abnormal data or the training data that may degrade the performance of the baseline network, ensuring effective feature extraction from the raw dataset and enhancing the validity of the data. This helps to improve the accuracy of the prediction results, and address the technical issues in the existing technologies where effective features from the raw dataset cannot be effectively extracted, leading to poor measurement accuracy.

The above describes the embodiments of a water quality measurement method provided by the present application. The description below gives the embodiments of a water quality measurement device provided by this application.

Please refer to FIG. 12, the water quality measurement device provided in the embodiments of this application includes:
Data acquisition unit, which is used for acquiring multiple sets of training data and determining the corresponding labels for each set. Here, the training data includes nitrate nitrogen concentration, dissolved oxygen concentration, influent total nitrogen concentration, and influent suspended solids concentration, and the labels are effluent ammonia nitrogen concentration or effluent total nitrogen concentration;
Training unit, which is used for iteratively training a baseline network according to the training data, and calculating the error based on the predicted values output by the baseline network and the corresponding labels;
Parameter update unit, configured to calculate the error state value based on the errors from two consecutive iterations. If the error state value of the current iteration satisfies the preset conditions, then the network parameters of the baseline network will be updated using the error of the current iteration. If the error state value does not meet the preset conditions, the network parameters will not be updated. This process continues until the baseline network converges and the measurement model is obtained;
Measurement unit, which is used for performing water quality measurement for the wastewater treatment plant based on the measurement model and obtaining the water quality measurement results.

As a further improvement, the device also includes:
Network construction unit, configured to construct the baseline network, which includes an input layer, a membership function layer, a fuzzy rule layer, a normalization layer, and an output layer;
Training unit, specifically used for:
Inputting the training data into the input layer of the baseline network;
Calculating the membership degree of the training data through the membership function layer;
Performing fuzzy processing on the membership degree of the training data through the fuzzy rule layer to obtain the fuzzy features of the training data;
Normalizing the fuzzy features of the training data through the normalization layer to obtain the normalized fuzzy features;
Performing the defuzzification of the normalized fuzzy features of the training data through the output layer to output the predicted values of the training data;
Calculating the errors based on the predicted values output by the baseline network and the corresponding labels.

As a further improvement, the measurement unit is specifically used for:
Collecting the water quality parameters of the wastewater treatment plant;
Inputting water quality parameters into the measurement model to predict the effluent ammonia nitrogen concentration or effluent total nitrogen concentration, thereby obtaining the water quality measurement results of the wastewater treatment plant.

In the embodiments of this application, when iteratively training the baseline network using training data, the error state value is derived based on the errors from each iteration. And the error state value determines whether to update the network parameters or not. If the error state value does not satisfy the preset conditions, the network parameters will not be updated. In this way, it prevents the backpropagation of the abnormal data or the training data that may degrade the performance of the baseline network, ensuring effective feature extraction from the raw dataset and enhancing the validity of the data. This helps to improve the accuracy of the prediction results, and address the technical issues in the existing technologies where effective features from the raw dataset cannot be effectively extracted, leading to poor measurement accuracy.

The embodiments of the present application also provide an electronic equipment, which includes a processor and a memory;
The memory is used to store program code and transmit the program code to the processor;
The processor executes the water quality measurement method from the aforementioned method embodiments based on the instructions in the program code.

Additionally, the embodiments of this application provide a computer-readable storage medium, which is used to store program code. When the program code is executed by a processor, it can implement any of the water quality measurement methods described in the aforementioned method embodiments.

Those skilled in the art shall clearly understand that, for the sake of convenience and brevity, the specific working process of the aforementioned devices and units can be referred to the corresponding processes in the previously described methods and embodiments, and will not be repeated here.

1. An electronic equipment, characterized in that the electronic equipment includes a processor and a memory;
the memory is configured to store and transmit instructions to the processor;
the processor is configured to execute the instructions to conduct a water quality measurement method, wherein the water quality measurement method comprises:
collecting water quality parameters from a wastewater treatment plant;
inputting the water quality parameters into a measurement model to predict an effluent ammonia nitrogen concentration or an effluent total nitrogen concentration corresponding to the water quality parameters, to obtain water quality measurement results of the wastewater treatment plant;
wherein the water quality measurement method further comprises a step of training the measurement model, the step of training the measurement model comprising:
acquiring multiple sets of training data and determining a label for each training data set, each training data set includes nitrate nitrogen concentration, dissolved oxygen concentration, influent total nitrogen concentration, and influent suspended solids concentration, with the label being either an effluent ammonia nitrogen concentration label or an effluent total nitrogen concentration label;
training a baseline network iteratively using the training data, and calculating an error based on a predicted value output by the baseline network and a corresponding label;
calculating an error state value based on the error obtained from a current iteration and a previous iteration of the current iteration, if the error state value of the current iteration satisfies the preset conditions, then network parameters of the baseline network is updated using the error of the current iteration, if the error state value does not meet the preset conditions, the network parameters isn't updated, this process continues until the baseline network converges and the measurement model is obtained;
the method also includes:
constructing the baseline network, which comprises an input layer, a membership function layer, a fuzzy rule layer, a normalization layer, and an output layer; the iterative training of the baseline network includes:
inputting the training data into the input layer of the baseline network;
calculating a membership degree of the training data through the membership function layer;
performing fuzzy processing on the membership degree of the training data through the fuzzy rule layer to obtain fuzzy features of the training data;
normalizing the fuzzy features of the training data through the normalization layer to obtain normalized fuzzy features;
performing a defuzzification of the normalized fuzzy features of the training data through the output layer to output the predicted values of the training data;
the calculation of the error state value based on the errors obtained from the current iteration and the previous iteration of the current iteration includes:
calculating a deviation between an error obtained in the current iteration and an error obtained in the previous iteration to derive a first error state value for the current iteration;
calculating a difference between the first error state value of the current iteration and the first error state value of the previous iteration to obtain a second error state value for the current iteration;
the process of determining whether the error state value of the current iteration satisfies the preset conditions includes:
determining whether both the first error state value and the second error state value of the current iteration are smaller than the preset threshold, when both the first error state value and the second error state value of the current iteration are smaller than the preset threshold, it can be concluded that the error state value of the current iteration satisfies the preset conditions, when both the first error state value and the second error state value of the current iteration aren't smaller than the preset threshold, it means that the error state value of the current iteration does not satisfy the preset conditions;
the process of updating the network parameters of the baseline network using the error from the current iteration includes:
calculating a learning rate for the current iteration based on the error of the current iteration;
using the learning rate and a gradient of the current iteration to update the network parameters of the baseline network, an update formula for the network parameters of the baseline network is:
$$〈 \begin{matrix}{Event}_{4} : Ω ( t + 1 ) = Ω ( t ) - ( Q ( t ) + λ ( t ) ) \cdot g ( t ) \\ otherwise : Ω ( t + 1 ) = Ω ( t )\end{matrix} \qquad ( 15 )$$
where Ω(t+1) is an updated network parameters of the baseline network, which includes a center c, width σ, feedback weights β, and output weights ω; Q(t) is a pseudo-Hessian matrix at a t-th iteration, g(t) is a gradient vector at the t-th iteration, and λ(t) is the learning rate at the t-th iteration; Event₄ denotes that the error state value at the current iteration t satisfies the preset conditions, while Otherwise indicates that the error state value at the current iteration t does not satisfy the preset conditions;
the calculation of the learning rate for the current iteration based on the error obtained from the current iteration includes:
calculating an L1-norm and L2-norm of the error of the current iteration;
performing a weighted sum of the L1-norm and L2-norm of the error based on preset condition parameters to obtain the learning rate for the current iteration.

2-4. (canceled)

5. A computer-readable storage medium, characterized in that it is used to store program code, when the program code is executed by a processor, it can implement a water quality measurement method, wherein the water quality measurement method comprises:
collecting water quality parameters from a wastewater treatment plant;
inputting the water quality parameters into a measurement model to predict an effluent ammonia nitrogen concentration or an effluent total nitrogen concentration corresponding to the water quality parameters, to obtain water quality measurement results of the wastewater treatment plant;
wherein the water quality measurement method further comprises a step of training the measurement model, the step of training the measurement model comprising:
acquiring multiple sets of training data and determining a label for each training data set, each training data set includes nitrate nitrogen concentration, dissolved oxygen concentration, influent total nitrogen concentration, and influent suspended solids concentration, with the label being either an effluent ammonia nitrogen concentration label or an effluent total nitrogen concentration label;
training a baseline network iteratively using the training data, and calculating an error based on a predicted value output by the baseline network and a corresponding label;
calculating an error state value based on the error obtained from a current iteration and a previous iteration of the current iteration, if the error state value of the current iteration satisfies the preset conditions, then network parameters of the baseline network is updated using the error of the current iteration, if the error state value does not meet the preset conditions, the network parameters isn't updated, this process continues until the baseline network converges and the measurement model is obtained;
the method also includes:
constructing the baseline network, which comprises an input layer, a membership function layer, a fuzzy rule layer, a normalization laver, and an output layer; the iterative training of the baseline network includes:
inputting the training data into the input layer of the baseline network;
calculating a membership degree of the training data through the membership function layer;
performing fuzzy processing on the membership degree of the training data through the fuzzy rule layer to obtain fuzzy features of the training data;
normalizing the fuzzy features of the training data through the normalization layer to obtain normalized fuzzy features;
performing a defuzzification of the normalized fuzzy features of the training data through the output layer to output the predicted values of the training data;
the calculation of the error state value based on the errors obtained from the current iteration and the previous iteration of the current iteration includes:
calculating a deviation between an error obtained in the current iteration and an error obtained in the previous iteration to derive a first error state value for the current iteration;
calculating a difference between the first error state value of the current iteration and the first error state value of the previous iteration to obtain a second error state value for the current iteration;
the process of determining whether the error state value of the current iteration satisfies the preset conditions includes:
determining whether both the first error state value and the second error state value of the current iteration are smaller than the preset threshold, when both the first error state value and the second error state value of the current iteration are smaller than the preset threshold, it can be concluded that the error state value of the current iteration satisfies the preset conditions, when both the first error state value and the second error state value of the current iteration aren't smaller than the preset threshold, it means that the error state value of the current iteration does not satisfy the preset conditions;
the process of updating the network parameters of the baseline network using the error from the current iteration includes:
calculating a learning rate for the current iteration based on the error of the current iteration;
using the learning rate and a gradient of the current iteration to update the network parameters of the baseline network, an update formula for the network parameters of the baseline network is:
$$〈 \begin{matrix}{Event}_{4} : Ω ( t + 1 ) = Ω ( t ) - ( Q ( t ) + λ ( t ) ) \cdot g ( t ) \\ otherwise : Ω ( t + 1 ) = Ω ( t )\end{matrix} \qquad ( 15 )$$
where Ω(t+1) is an updated network parameters of the baseline network, which includes a center c, width σ, feedback weights β, and output weights ω; Q(t) is a pseudo-Hessian matrix at a t-th iteration, g(t) is a gradient vector at the t-th iteration, and λ(t) is the learning rate at the t-th iteration; Event₄ denotes that the error state value at the current iteration t satisfies the preset conditions, while Otherwise indicates that the error state value at the current iteration t does not satisfy the preset conditions;
the calculation of the learning rate for the current iteration based on the error obtained from the current iteration includes:
calculating an L1-norm and L2-norm of the error of the current iteration;
performing a weighted sum of the L1-norm and L2-norm of the error based on preset condition parameters to obtain the learning rate for the current iteration.
